(19) United States
(12) Patent Application Publication
(10) Pub. No.: US 2026/0060604 A1
(43) Pub. Date: Mar. 5, 2026

(54) Wearable ring with microfluidic perspiration collector and dock-based optical biomarker analysis

(71) Applicant: Senbiosys, Neuchâtel (CH)

(72) Inventors: Assim Boukhayma, Neuchâtel (CH); Antonino Caizzone, Milvignes (CH)

(21) Appl. No.: 19/318,205

(22) Filed: Sep. 3, 2025

Related U.S. Application Data
(60) Provisional application No. 63/690,467, filed on Sep. 4, 2024.

(51) Int. Cl. A61B 5/00; A61B 5/024; A61B 5/1455; A61B 5/1486

(52) U.S. Cl. CPC A61B 5/4266; A61B 5/02433; A61B 5/1455; A61B 5/1486; A61B 5/6802; A61B 2560/0456; A61B 2562/04; A61B 2562/166

(57) ABSTRACT

A wearable smart ring includes an annular body sized to receive a finger, photoplethysmography (PPG) sensors arranged along an inner peripheral surface, and a microfluidic perspiration collector disposed at the inner surface. The collector has microchannels that draw perspiration by capillary action into one or more reservoirs and includes a region that concentrates or retains biomarkers. A docking station mechanically receives the ring for charging and interfaces with the collector to transfer fluid and/or perform in-situ analysis. The dock includes optical instrumentation (e.g., photometry, spectroscopy such as Raman) and may implement pumping and flushing to reset the collector for subsequent use. A controller communicates results by wireless links. The system enables passive collection of perspiration during wear and detailed biomarker analysis during charging, thereby augmenting PPG-derived physiological measurements with perspiration chemistry without burdening ring power or size.

### RELATED APPLICATIONS

This application claims the benefit under 35 USC 119(e) of U.S. Provisional Application No. 63/690,467, filed on Sep. 4, 2024, which is incorporated herein by reference in its entirety.

### BACKGROUND OF THE INVENTION

Nowadays, wearable devices, such as fitness trackers or smartwatches, with optical heart rate sensors, are becoming common. Even rings are now available with these sensors.

The technology behind these sensors is called photoplethysmography (PPG), which is an optical measurement technique used to detect blood volume changes in living tissues. A PPG sensor requires a few optoelectronics components, such as a light source, e.g. light-emitting-diode (LED), to illuminate the living tissue, a photodetector (PD) to track any light intensity variation due to the blood volume change, and an analog front-end (AFE) for signal conditioning and processing. Today, the importance of PPG for medical monitoring is proven by the number of primary vital signs directly or indirectly that can be resolved by it.

In a typical example, the PPG signal is obtained by shining light from the LED at a given wavelength, in the visible or near-infrared range, into the finger, wrist, forehead, or ear lobes. The PPG sensor's photodetector detects the light transmitted through (transmissive PPG) or reflected (reflective PPG) from the tissue and transforms it into a photogenerated current. The detected signal, i.e. PPG signal, has two different components: a large DC (quasi-static) component corresponding to the light diffusion through tissues and non-pulsatile blood layers, and a small AC (pulsatile) part due to the diffusion through the arterial blood. The AC component is only a very small fraction (typically 0.2% to 2%) of the DC one, meaning the AC component is 500 to 50 times smaller than the DC component. This AC/DC ratio mostly depends on the sensor's location on the body, and the LED wavelength, and weakly on the skin tone. This AC/DC ratio is often called perfusion-index (PI) and ultimately sets one of the challenges for any PPG readout system. Indeed, the AC component carries most of the biomedical information. Low PI values lead to reduced signal fidelity, complicated signal processing schemes and larger power consumption.

U.S. Pat. Pub. No. US 2023/0008487, entitled Sensing System and Method for Smart Rings Employing Sensor Spatial Diversity, by the inventors hereof, describes devices and operating methods allowing a better photoplethysmographic sensing on the finger or other digit or other location where transmissive PPG and/or reflective PPG is possible. These described devices can enable lower power consumption, higher fidelity, and/or greater versatility to different use cases and users' specificities. This PPG system takes advantage of sensor spatial diversity to enhance the quality and the reliability of the PPG measurements in smart rings, for example.

At the same time, perspiration, or sweating, is a natural bodily function that plays a crucial role in thermoregulation, helping to maintain a stable internal body temperature. When the body's temperature rises—due to external heat or physical exertion—the sweat glands, primarily the eccrine glands, are stimulated to release moisture as perspiration or sweat. This moisture evaporates from the skin's surface, cooling the body through heat loss.

Perspiration is composed mostly of water, but it also contains a variety of solutes that make it a valuable source of biomarkers for health monitoring. Among the primary components are electrolytes such as sodium, potassium, calcium, and magnesium, which are critical for maintaining cellular function and overall fluid balance within the body. The concentration of these electrolytes in perspiration can provide insights into hydration levels and electrolyte balance, which are particularly important in clinical and athletic contexts.

Additionally, perspiration contains small amounts of metabolic waste products like urea, lactate, and ammonia. The levels of these compounds can reflect metabolic and physiological conditions. For instance, elevated lactate levels in perspiration can indicate intense physical activity or conditions that affect metabolism. Researchers are also exploring the use of other substances found in perspiration, such as glucose, hormones like cortisol, and environmental toxins, as indicators of health status or exposure to harmful substances.

### SUMMARY OF THE INVENTION

The analysis of these biomarkers through non-invasive perspiration testing offers potential for real-time health monitoring and disease diagnosis. Innovations in wearable technology will increasingly enable the measurement of perspiration composition, providing valuable data for managing various health conditions, tracking athletic performance, and even detecting early signs of diseases. As the understanding of the composition of perspiration and its relationship to health continues to evolve, it could greatly enhance personalized medicine approaches and health monitoring technologies analyze the perspiration, augmenting the other information offered by PPG sensing.

Different measurement sites can be used including the wrist, the finger and the ear region (both the lobe and the canal). The finger, however, has been shown to be among the best locations in terms of PI, at a given optical power, providing better biomedical sensing. In addition, perspiration is also present here.

The present invention concerns a sensor ring system with a microfluidic biomarker collector. Perspiration is absorbed and at least biomarkers are retained by the collector for later analysis at a docketing station.

The fingers are a good location for spontaneous perspiration collection. Perspiration is generated at fingertips skin continuously without need for stimulation. The present system often employs microfluidics to collect perspiration and especially the biomarkers contained in the perspiration, which are retained in the collector.

In general, according to one aspect, the invention features a wearable ring comprises an annular body defining an inner peripheral surface sized to receive a finger; one or more photoplethysmography (PPG) sensor units arranged around the inner peripheral surface; and a microfluidic perspiration collector disposed at the inner peripheral surface. The collector includes intake microchannels dimensioned to wick perspiration by capillary action into at least one reservoir during wear, and an interface region configured, when the ring is seated in a docking station, to provide at least one of: (i) an optical path through an optical window for interrogation of perspiration or biomarkers present in the collector, or (ii) a fluidic coupling for transfer of perspiration or retained biomarkers from the collector to the docking station for analysis.

In certain embodiments, the PPG sensor units are operated in reflective and/or transmissive PPG modes under control of a controller on the ring. At least one PPG sensor unit can include a photodiode array and two light emitters that emit at different wavelengths in the visible and/or infrared, and the PPG readout electronics may be embedded within the photodiode array.

In some embodiments, the collector comprises inlet pores opening at the inner peripheral surface to feed the intake microchannels. Surfaces of the intake microchannels may be rendered hydrophilic, and at least one hydrophobic capillary stop valve may be patterned to control filling. The collector can include a retention region comprising a cation- or anion-exchange medium configured to concentrate at least one of sodium, potassium, calcium, or magnesium; an enzymatic reaction pad; and/or an affinity capture layer for cortisol or an environmental toxin. Non-reactive channel surfaces may be coated with an anti-fouling layer.

The interface region's optical window can be formed of PMMA or COC and positioned by alignment features for repeatable coupling to optics of the docking station. In some versions, the interface region includes a fluidic service port separate from the window; in other versions, the interface region is configured so that the window and the fluidic coupling are co-located or integrated, enabling optical interrogation and/or transfer through the window when the docking station mates to the ring with a compliant seal.

The intake microchannels may have widths of about 5-200 μm, heights of about 5-100 μm, and lengths of about 1-50 mm, and the at least one reservoir may have a volume of about 0.5-10 μL. The collector may further include hydrophobic vents that permit vapor equilibration while inhibiting liquid leakage toward electronics.

In another aspect, the invention features a method of analyzing perspiration using the wearable ring and a docking station comprises: placing the wearable ring on a user's finger; during wear, passively collecting perspiration in the microfluidic collector via the intake microchannels into the at least one reservoir; after wear, seating the ring in the docking station with the interface region aligned to the docking station; performing an analysis selected from (i) optically interrogating perspiration or biomarkers through the optical window of the interface region while the fluid remains in the collector, and (ii) transferring at least a portion of the perspiration or retained biomarkers through a fluidic coupling from the collector to an analyzer of the docking station and analyzing the transferred sample; and resetting the collector after the analysis by at least one of a rinse, a pressure pulse, air purging, or heating to prepare the collector for a subsequent collection cycle.

In a further aspect, a biosensing system comprises: (a) a wearable ring having an annular body defining an inner peripheral surface and including one or more of PPG sensor units and a microfluidic perspiration collector disposed at the inner peripheral surface, the collector collecting perspiration by capillary action into at least one reservoir and providing a window for fluidic and/or optical access; and (b) a docking station configured to receive the ring in a defined orientation and including an analyzer having an optical transmitter and an optical detector aligned to the interface region, and a controller programmed to perform at least one of: (i) optical analysis through the window while fluid remains in the collector, or (ii) transfer of perspiration or retained biomarkers from the collector through the window to an internal flow cell for analysis, wherein the docking station is further configured to reset the collector after analysis by at least one of a rinse, a pressure pulse, or heating.

In yet another aspect, a replaceable microfluidic perspiration collector is configured for installation at an inner peripheral surface of a wearable ring. The collector includes: (a) an intake region having pores that open to the inner peripheral surface; (b) a network of microchannels dimensioned to passively wick perspiration by capillary action to at least one reservoir; and (c) a functional region positioned adjacent to an optical window or a fluidic service port, the functional region including at least one of (i) a retention medium selected from ion-exchange material, affinity capture material, or a molecularly imprinted polymer, or (ii) an indicator or enzyme pad that produces an optical response to a target biomarker; wherein the collector is configured to be interrogated optically through the optical window or to release fluid through the service port when engaged by a docking station.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a side cross-sectional schematic view perpendicular to the axis of a sensor ring system, showing one example of a smart ring with a microfluidic biomarker collector;

FIG. 2 is a side cross-sectional schematic view perpendicular to the axis of a sensor ring system, showing another example of a smart ring with a microfluidic biomarker collector;

FIG. 3 is a schematic plan view of a PPG sensor 200 for the smart ring;

FIG. 4 is a schematic plan view of the smart ring and docking station with a collected biomarker analyzer; and

FIG. 5 is a flow diagram showing a method of operation of the smart ring and docking station.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

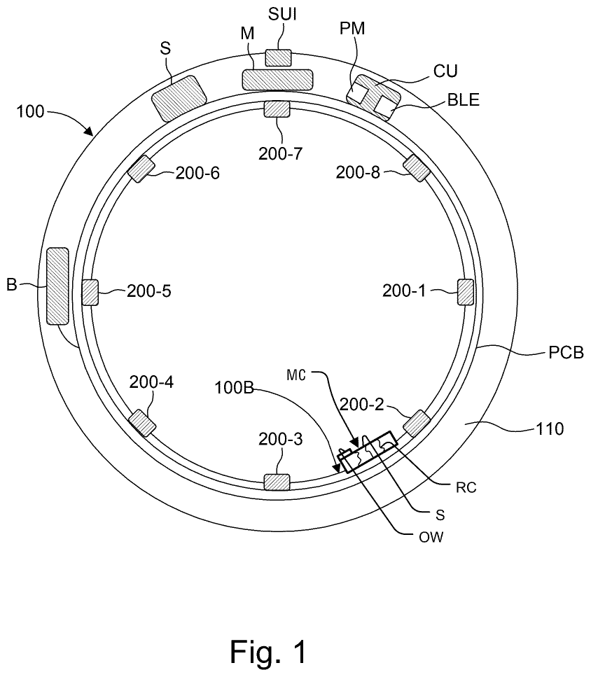

FIG. 1 shows an example of the sensor ring system 100.

In general, the sensor ring includes a ring or annual-shaped ring body 110 having an inner bore 110B sized to receive a human digit such as a finger, and specifically an index finger, middle finger, ring finger, or little finger or pinkie. The ring body 110 is typically a molded plastic or ceramic material, but could also be metal.

An array of PPG sensor units 200-1 to 200-8 are distributed around the inner bore and directed inwardly in order to perform PPG sensing of the digit inserted into the inner bore 110B. In the illustrated example, the sensor units 200-1 to 200-8 are evenly arrayed in a ring concentric to the ring body 110 and mounted to a flexible printed circuit board PCB that is affixed to an inner side facing into the inner bore 110B.

A control unit CU is further installed on the PCB. The control unit CU controls the triggering of and readout from the PPG sensors 200. In one mode of operation, the control unit CU alternates between a transmissive PPG mode and reflective PPG mode. In one example, the control unit controls the sensor units 200 to perform a combination of transmissive and reflective PPG sensing as described in U.S. Pat. Appl. Publ. No. US-2023-0008487 A1, which is incorporated herein by this reference in its entirety.

The control unit CU preferably includes a Bluetooth-low-energy communications block BLE and a power management block PM, for communicating data with the external world and regulating system power, respectively.

The control unit CU stores sensor readings to a memory unit M also on the PCB. The memory unit also contains the operating instructions for the system set-up and other firmware components required for operation.

A battery B provides power to the system via the PCB, and is generally controlled by the power management unit PM in order to provide for extended operation.

A sensor block S has inertial sensors, including a three axis accelerometer and a three axis gyroscope. In some cases, the accelerometer and the gyroscope each have more than three axes, possibly six axes. In addition, the sensor block S also preferably has a user temperature sensor and an ambient temperature sensor, for separate body and environmental temperature detection.

A smart user interface block SUI provides active feedback from the device to the user and also receives user control input. In some examples, the SUI block includes a visible LED array and a touch sensor system, such as a capacitive touch sensor array.

According to the invention, a microfluidic biomarker collector MC is located in the inner bore 100B of the ring 100. In some examples, it collects perspiration. In other examples, perspiration flows through the microfluidic biomarker collector, which then retains only the biomarkers. Exemplary biomarkers retained by the collector MC include sodium, potassium, calcium, magnesium, urea, lactate, ammonia, glucose, hormones (e.g., cortisol), and environmental toxins.

In some examples, the microfluidic biomarker collector MC is a passive device that simply collects the biomarkers of interest. In other examples, the collector is an active device that is powered via the PCB and is controlled by the control unit CU.

In many implementations, the collector MC comprises of a network of microfluidic channels and reservoirs RC embedded within the structure of the ring. These microchannels are engineered to induce capillary action, drawing perspiration away from the skin and into tiny analysis fluidic paths for storage and possibly on-board analysis.

The surface S of the collector that contacts the skin features a series of microscopic pores or openings that allow perspiration to enter the microfluidic system of the collector MC. Once inside, the perspiration travels through the narrow, labyrinthine channels RC, propelled by both capillary action and siphoning mechanisms designed into the flow paths. This design ensures that even small amounts of perspiration can be effectively captured and transported for biomarker capture and/or concentration.

In some examples, within the collector MC, different sections of the microfluidic channels are lined with various sensor technologies that are interrogated by the control unit CU. These sensors are tailored to detect specific biomarkers such as electrolytes (sodium, potassium), metabolic byproducts (lactate, urea), and even more complex molecules like hormones or environmental pollutants. The integration of these sensors into the microfluidic architecture allows for real-time and/or later analysis of composition, providing feedback via a connected app on a smartphone or other device.

In some embodiments, surface S of the microfluidic collector MC that contact perspiration are rendered hydrophilic to promote spontaneous capillary wicking. Suitable treatments include oxygen plasma or UV-ozone activation of polymeric channel walls (e.g., PC, PMMA, COC/COP blends, or parylene-coated ceramics), optionally followed by grafting of polar monolayers or thin films (e.g., silanols, polydopamine, or polyethylene glycol (PEG) derivatives) to stabilize the contact angle over time. Hydrophobic regions are patterned as capillary stop valves or vents by masking during activation and/or depositing a low-surface-energy coating (e.g., fluorosilane or fluoropolymer). The resulting hydrophilic/hydrophobic patterning defines intake pores, metering sections, and reservoirs that fill passively during wear and remain stable against backflow until docking.

To retain or concentrate electrolytes, at least one section of the channels and reservoirs RC of the collector MC includes a cation- or anion-exchange medium. Examples include sulfonated or carboxylated polymer membranes or beads embedded in a hydrogel matrix positioned within a pocket of the channel. The fixed charges produce a Donnan potential and selective uptake of target ions (e.g., Na⁺, K⁺, Ca²⁺, Mg²⁺), increasing local concentration relative to water. The pocket is preferably located adjacent to an optical and fluidic window OW of the ring so that, when docked, the station's analyzer interrogates the retained ions directly or after a controlled elution step. In some cases, optically responsive ionophores or chromoionophores are immobilized in this region to transduce local ion activity into an absorbance or fluorescence change readable through the window.

For metabolic biomarkers (e.g., lactate, urea, ammonia, glucose), the collector MC incorporates one or more reaction pads in the channels and reservoirs RC in embodiments comprising immobilized enzymes and optionally co-factors or chromogenic/fluorogenic indicators. Illustrative pads include: lactate oxidase (with peroxidase/indicator couple), urease, or glucose oxidase immobilized within a cross-linked hydrogel or porous scaffold. The pads are positioned downstream of the intake so that perspiration rehydrates the reagents during wear. Colorimetric or fluorescent products accumulate within a defined optical path length, enabling quantitative readout by the docking station. Reagent loading can be lyophilized for shelf stability and regenerated or replaced during the dock's flush/reset cycle, depending on the chemistry chosen.

In certain embodiments, a retention region of the channels and reservoirs RC is functionalized with affinity capture chemistry for neutral or weakly ionic biomarkers such as cortisol or selected environmental toxins. Capture can be achieved using polymer brushes, antibodies, molecularly imprinted polymers, or nucleic-acid aptamers immobilized on the channel wall or a microporous insert. Immobilization routes include silane coupling to oxide/glass features, thiol-to-metal attachment on a sputtered thin film, or polydopamine primer layers on polymer. In some versions, the affinity layer is labeled with a fluorophore/quencher pair or a ratiometric dye whose signal changes upon target binding, allowing in-situ optical interrogation through the ring window W; in other versions, the dock performs a competitive elution and analyzes the eluate spectroscopically.

To reduce nonspecific adsorption and preserve channel permeability, non-reactive surfaces are coated with anti-fouling layers such as PEG, zwitterionic polymers, or hydrophilic parylenes. Skin-contact features are formed from ISO-10993-compliant materials. Barrier coatings and gasketed joints isolate the fluidic volume from electronics. Where vents are provided, hydrophobic membranes permit vapor equilibration while preventing leakage.

The functional regions described above are arranged along the flow path of the channels and reservoirs RC from the intake pores in the surface at the inner peripheral surface S to the optical and fluidic window OW adjacent to the station interface. A representative sequence is: (i) hydrophilic intake and metering channel; (ii) ion-exchange pocket; (iii) optional enzymatic pad; and (iv) optical window with an affinity layer or indicator film. Dimensions may include channel widths of about [5-200 μm], heights of about [5-100 μm], and reservoir volumes of about [0.5-10 μL] per sector; these values are exemplary and non-limiting.

The collector MC is fabricated in certain embodiments by laminating patterned polymer films or micromolding a single body, then locally treating surfaces through masked plasma/UV steps to define hydrophilic and hydrophobic zones. Functional reagents are dispensed by inkjet or micro-pipette into designated pockets and cured or lyophilized. An optical window (e.g., PMMA or COC) is bonded over the reaction/retention region, and alignment features position this window relative to the docking station optics and fluid openings. Assemblies are sealed, stored with desiccant, and integrated into the ring body.

The control unit CU analyses the sensor data and any analysis performed by the microfluidic collector MC, translates it into meaningful health metrics, and communicates wirelessly with external devices for further analysis or immediate display of results.

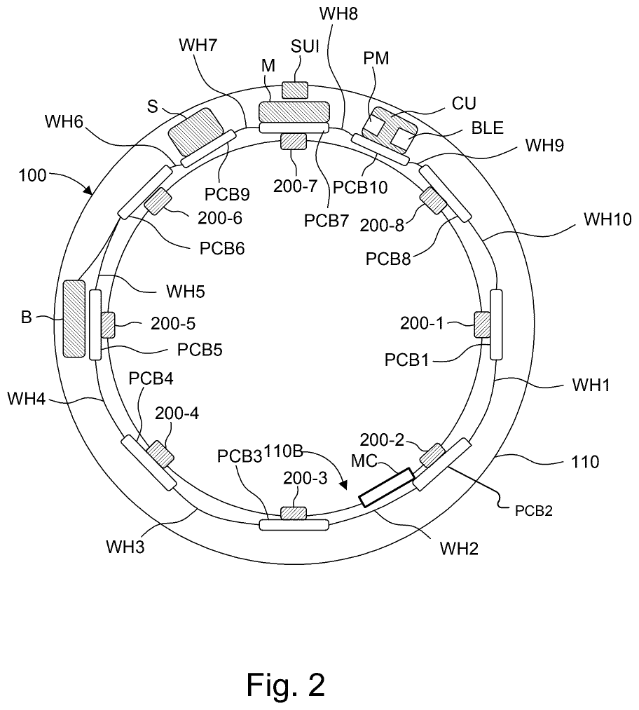

FIG. 2 shows another example of the sensor ring system 100. This uses a series of small independent PCBs, i.e., PCB1-PCB10 for each of the sensor units 200, the battery B, the memory M and smart user interface block SUI, the control unit CU, and sensor block S. Each of these PCBs are interconnected with wiring harnesses WH1-WH10 in a daisy-chained fashion. The collector MC as described in connection with FIG. 1 is integrated with PCB2 in the illustrated example.

In still other examples, the independent PCBs are replaced with one or more flexible PCBs. In both cases, flexible PCBs can be used to allow a better integration into the system.

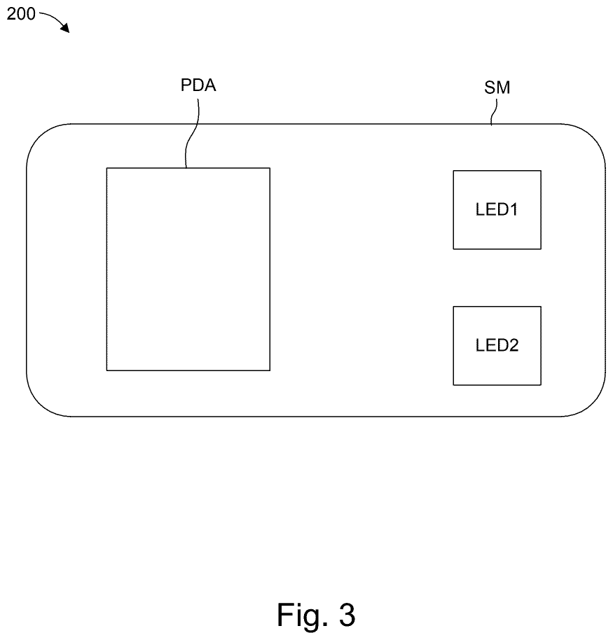

FIG. 3 shows an exemplary configuration for each of the PPG sensors 200.

In a typical implementation, each PPG sensor 200 includes at least one and preferably multiple photodiodes. In one example, an array of at least 8 by 8 photodiodes PDA is provided on a submount SM.

Each sensor 200 also includes one or more light emitters LED1, LED2 such as light emitting diodes (LED) or vertical cavity surface emitting lasers (VCSELs) installed on the submount SM, next to a light sensor such as a photodiode or photodiode array PDA, also on the submount. Moreover, the light emitters LED1, LED2 can operate at different wavelengths. In the illustrated example, two light emitters LED1 and LED2 are provided on the submount SM that emit at different wavelength in the visible and/or infrared.

In a current example, to reduce the PPG sensor size, the PPG readout electronics is embedded into the array of photo diodes PDA.

Incorporated U.S. Pat. Pub. No. US 2023/0008487 entitled Sensing System and Method for Smart Rings Employing Sensor Spatial Diversity is incorporated herein in its entirety. This additionally describes methods of operation of the ring system 100 including solutions for processing reflective and transmissive PPG sensing operations.

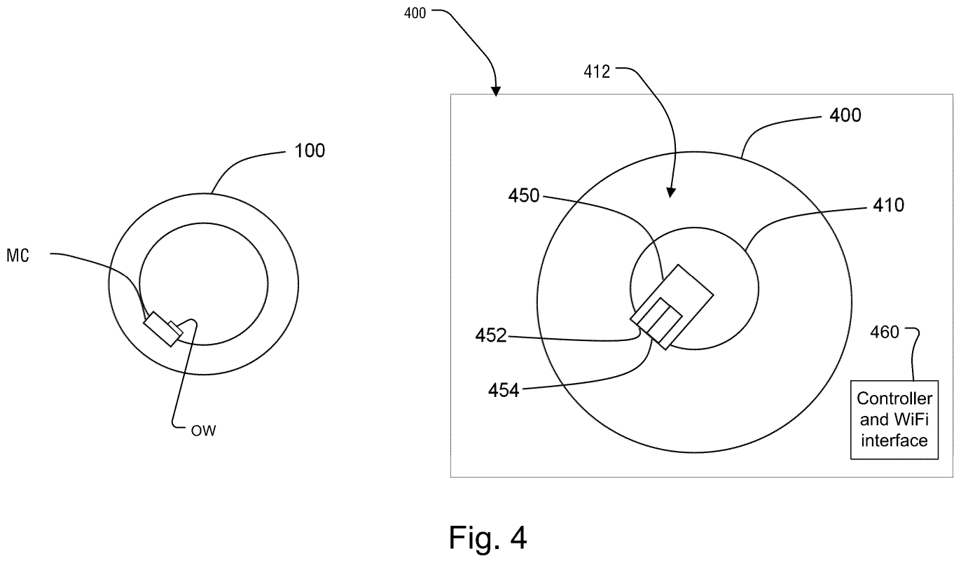

FIG. 4 shows the smart ring 100 and a docking station 400 with a collected biomarker analyzer 450 (“CBA 450”).

The docking station 400 is designed for analyzing the biomarkers collected by the microfluidic perspiration collector MC of the ring 100 and to complement the ring's capabilities. The station 400 serves as both a charging dock and an analyzer, allowing for detailed examination of perspiration composition captured by the ring's collector MC.

The docking station preferably has a sleek, compact design that can easily fit on a nightstand or a desk. When the ring 100 is placed into the annular depression 412 formed into a top surface of the docking station 400, the collector MC aligns with the CBA optics—specifically the optical transmitter 452 and optical detector 454—and, in some embodiments, with a fluidic interface for transfer and flushing.

The MC and the CBA 450 together implement a distributed assay system in which collection occurs during wear and most or all quantitative analysis occurs during docking. The distribution of functions may vary along a continuum from: (i) dock-centric analysis, where the MC primarily stores or pre-conditions fluid and the CBA performs measurements; to (ii) in-situ interrogation, where the MC hosts indicator or capture chemistries and the CBA provides optical readout only; to (iii) hybrid approaches in which the ring performs coarse screening and the dock executes confirmatory, higher-specificity assays.

In a dock-centric embodiment, the MC acts as a passive sampler and concentrator. During wear, perspiration wicks through hydrophilic intake microchannels into one or more reservoirs where retention media (e.g., ion-exchange membranes, affinity pads) concentrate target biomarkers. Upon docking, the CBA 450 establishes a fluidic and/or optical interface to the MC via the window W and actively transfers a fraction of the collected fluid and/or releases retained analytes to an internal flow cell. The CBA 450 may incorporate a micro-pump (peristaltic, piezoelectric diaphragm, electroosmotic, or syringe-style) and micro-valves to meter boluses of about [0.1-10 μL], mix with reagents, and drive the sample through optical paths of known length. Detection can include photometry, fluorimetry, and/or Raman spectroscopy using the transmitter 452 and detector 454 interfacing with the optical window OW. After analysis, the CBA applies a rinse and controlled pressure pulse to reset the MC for a subsequent collection cycle.

In an in-situ embodiment, the MC includes one or more indicator regions that transduce local analyte activity to an optical signal (e.g., chromoionophore films for Na⁺/K⁺, enzymatic color/fluorescence pads for lactate/urea, or ratiometric dye films for cortisol). The ring provides optical and fluidic window W adjacent to these regions. During docking, the CBA 450 aligns an excitation beam from 452 through the optical window OW and collects reflected/transmitted/fluorescent light at 454 without transporting fluid out of the MC. The CBA may acquire multiple wavelengths and perform ratiometric or kinetic fits to determine concentration. A short re-standardization rinse and/or optical baseline check can be performed while fluid remains in place.

In a hybrid embodiment, the MC supports two phases: (1) a low-power on-ring screen using indicator films or a miniature reflectance color sensor positioned under the optical window to estimate coarse levels or to detect saturation; and (2) a high-specificity dock assay that either re-reads the same region with higher signal-to-noise instrumentation or elutes the captured analyte via the optical and fluidic window OW into the CBA flow cell for confirmatory analysis. The ring may store screen results and context metadata (collection time, temperature, motion level) for use by the dock algorithms during calibration and trend analysis.

To achieve repeatable coupling, the ring 100 and dock 400 may include kinematic features such as a keyed annular depression 412, magnets, pins, or asymmetrical flats that index the MC window to the CBA optics within ±[50-200 μm]. Where fluid transfer is used, a compliant micro-gasket or septum mates to a port on the MC to form a leak-tight interface. Hydrophobic break regions in the MC near the port inhibit unintended drainage during wear. The CBA may monitor contact quality using a low-power alignment photodiode check or a pressure-decay test across the mated seal before initiating analysis.

The MC can be divided into multiple sectors circumferentially spaced around the inner bore (e.g., four sectors each with about 0.5-10 μL capacity). Each sector may target a different biomarker or chemistry. The CBA 450 sequentially interrogates sectors by rotational indexing of the ring within the depression 412 or by steering the optical path to each window. In transfer-type embodiments, the dock's micro-valve manifold selects the active sector port and routes the eluate to the desired internal assay channel.

In some embodiments, the MC includes minimal actuation for reliability and power conservation, such as a resistive micro-heater (≤100 mW, duty-cycled) to accelerate drying after docking or to trigger controlled release from a thermo-responsive hydrogel. Alternative actuation includes electroosmotic electrodes or a bistable capillary valve that opens only when the dock applies a threshold pressure. These features reduce dock time and improve cycle-to-cycle reproducibility while keeping average ring power low.

The CBA 450 may integrate interchangeable or parallel detector modules: (i) a broadband LED/photodiode photometer for absorbance (path length about [0.2-2 mm]); (ii) a fluorescence head with optical filters; and/or (iii) a ratiometric Raman module with two excitation wavelengths for internal referencing. The CBA controller executes assay scripts that specify: pre-wetting, blank measurement, sample read(s), reagent addition(s), kinetic monitoring, and reset. For electrochemical options, the CBA may host ion-selective electrodes or ISFETs in the internal cell, with the MC providing only collection and pre-concentration.

Following analysis, the CBA executes a reset routine: flush the MC with a specified rinse (e.g., DI water or buffer), optional air purge, low-power heating to drive off residual moisture, and a UV-LED exposure for hygiene. Hydrophobic vents in the MC allow vapor exchange while preventing leakage into electronics. Where the chemistry is not regenerable, the MC is implemented as a replaceable insert accessed through the inner bore.

In use, perspiration chemistry measured via the CBA is fused with PPG-derived metrics and inertial/temperature context from the ring. For example, electrolyte levels and collection rate can be used to adjust PPG LED drive or interpret perfusion-index changes under high perspiration. The control unit CU aggregates these data and synchronizes them with the CBA analysis results via BLE or Wi-Fi (through the dock's interface 460).

During docking, the station aligns the excitation/detection path with the window of the microcollector MC and, in some embodiments, applies a controlled pressure or flow pulse to (a) move fresh fluid across a functional region for kinetic measurements, (b) elute retained analytes for analysis in an internal flow cell, and/or (c) reset the collector. The station may also deliver a rinse or buffer droplet to re-standardize indicator films and to extend service life of enzymatic or affinity layers.

The analysis process is automated: once the ring 100 is docked, the CBA 450 pumps small volumes of perspiration and/or biomarkers from the ring's collector reservoirs through its internal analytical circuitry or performs in-situ optical analysis through the window. As the biomarkers are interrogated, sensors capture data on the presence and concentration of various biomarkers. This data is then processed using built-in algorithms designed to assess health metrics, track long-term trends, and potentially flag indicators of health issues.

Additionally, the docking station 400 is designed to be smart and connected. It can integrate seamlessly with home Wi-Fi networks, allowing its controller and Wi-Fi interface 460 to upload data to cloud-based health management systems or directly to healthcare providers. Users can interact with the station via a smartphone app or computer software to receive reports and visualizations.

This docking station not only enhances the functionality of the smart ring by providing detailed biomarker analysis but also adds convenience by ensuring the ring is charged and ready for continuous use. Its integration into personal and healthcare ecosystems makes it a valuable tool for proactive health monitoring and management.

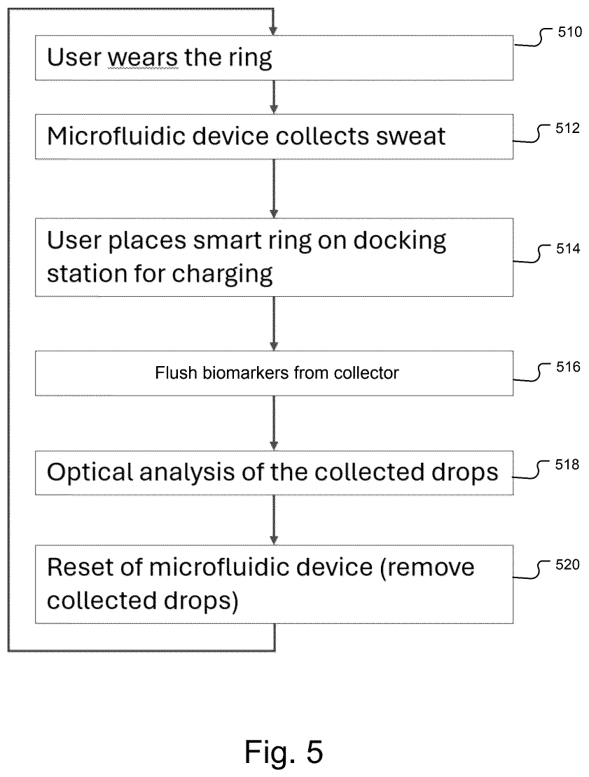

FIG. 5 is a flow diagram showing a method of operation of the smart ring and docking station.

The flow diagram outlines the sequence of operations for a health monitoring system that utilizes the smart ring 100 equipped with a microfluidic collector MC to collect and analyze perspiration. Here's a step-by-step description of the process illustrated in the diagram:

Step 510 involves the user wearing the smart ring on their finger. This ring contains the microfluidic collector MC that is designed to and adapted to collect perspiration and/or biomarkers from the skin.

As the user goes about their daily activities, the microfluidic collector MC within the ring 100 automatically collects perspiration and/or concentrated biomarkers in step 512. This device channels the perspiration through a network of tiny channels where it can be temporarily stored or prepared for analysis. In some embodiments, these channels have widths [5-200 μm], heights [5-100 μm], lengths [1-50 mm]; reservoir volume [0.5-10 μL] per sector.

After a certain period of use, or once enough perspiration and biomarkers have been collected, the user removes the ring 100 and places it onto a docking station 400 in step 514. This docking station serves dual purposes: it recharges the ring's battery and interfaces with the ring to sensor system 450 to initiate the analysis of collected perspiration by the operation of the station controller 460 as described.

The docking station often flushes the biomarkers from the collector into the sensor system in step 516 by the controller 460 instructing the sensor system 450 to initiate the analysis and then uses optical and other methods to analyze the perspiration in step 518 in the sensor system. In other examples, the sensor system 450 analyzes the biomarkers in the collector MC. These analysis methods could involve spectroscopy, photometry, or other optical sensors and chemical sensors to measure the concentration of various biomarkers within the perspiration. This step may be repeated as necessary to ensure thorough analysis.

After the analysis is complete, the microfluidic collector MC within the ring 100 is reset in step 520. This involves cleaning or further flushing out the collected perspiration to prepare the device for another cycle of collection and analysis.

This automated process provides a non-invasive, convenient, and efficient way for individuals to monitor various health metrics through perspiration analysis, integrating seamlessly into daily life with minimal disruption.

While this invention has been particularly shown and described with references to preferred embodiments thereof, it will be understood by those skilled in the art that various changes in form and details may be made therein without departing from the scope of the invention encompassed by the appended claims.

What is claimed is:

1. A wearable ring comprising:
an annular body defining an inner peripheral surface sized to receive a finger;
one or more photoplethysmography (PPG) sensor units arranged around the inner peripheral surface; and
a microfluidic perspiration collector disposed at the inner peripheral surface, the collector including intake microchannels dimensioned to wick perspiration by capillary action into at least one reservoir during wear, and an interface region configured, when the ring is seated in a docking station, to provide at least one of:
(i) an optical path through an optical window for interrogation of perspiration or biomarkers present in the collector, or
(ii) a fluidic coupling for transfer of perspiration or retained biomarkers from the collector to the docking station for analysis.

2. The wearable ring of claim 1, wherein the one or more PPG sensor units are operated in reflective and/or transmissive PPG modes under control of a controller on the ring.

3. The wearable ring of claim 1, wherein at least one PPG sensor unit includes a photodiode array and two light emitters that emit at different wavelengths in the visible and/or infrared.

4. The wearable ring of claim 3, wherein PPG readout electronics are embedded within the photodiode array.

5. The wearable ring of claim 1, wherein the microfluidic perspiration collector comprises inlet pores opening at the inner peripheral surface to feed the intake microchannels.

6. The wearable ring of claim 1, wherein surfaces of the intake microchannels are rendered hydrophilic and at least one hydrophobic capillary stop valve is patterned to control filling.

7. The wearable ring of claim 1, wherein the collector includes a retention region comprising a cation- or anion-exchange medium configured to concentrate at least one of sodium, potassium, calcium, or magnesium.

8. The wearable ring of claim 1, wherein the collector includes an enzymatic reaction pad.

9. The wearable ring of claim 1, wherein the collector includes an affinity capture layer for cortisol or an environmental toxin.

10. The wearable ring of claim 1, wherein non-reactive channel surfaces are coated with an anti-fouling layer.

11. The wearable ring of claim 1, wherein the optical window of the interface region is positioned by alignment features for repeatable coupling to optics of the docking station.

12. The wearable ring of claim 1, wherein the intake microchannels have widths of about 5-200 μm, heights of about 5-100 μm, and lengths of about 1-50 mm, and the at least one reservoir has a volume of about 0.5-10 μL.

13. The wearable ring of claim 1, wherein the collector includes hydrophobic vents that permit vapor equilibration while inhibiting liquid leakage toward electronics.

14. A method of analyzing perspiration using the wearable ring and a docking station, the method comprising:
placing the wearable ring on a user's finger;
during wear, passively collecting perspiration in the microfluidic collector via the intake microchannels into the at least one reservoir;
after wear, seating the ring in the docking station with the interface region aligned to the docking station;
performing an analysis selected from:
(i) optically interrogating perspiration or biomarkers through the optical window of the interface region while the fluid remains in the collector, and
(ii) transferring at least a portion of the perspiration or retained biomarkers through a fluidic coupling from the collector to an analyzer of the docking station and analyzing the transferred sample; and
resetting the collector after the analysis by at least one of a rinse, a pressure pulse, air purging, or heating to prepare the collector for a subsequent collection cycle.

15. A biosensing system comprising:
a wearable ring having an annular body defining an inner peripheral surface and including one or more of PPG sensor units and a microfluidic perspiration collector disposed at the inner peripheral surface, the collector collecting perspiration by capillary action into at least one reservoir and a window for a fluidic and/or optical access; and
a docking station configured to receive the ring in a defined orientation and including an analyzer having an optical transmitter and an optical detector aligned to the interface region, and a controller programmed to perform at least one of:
(i) optical analysis through the window while fluid remains in the collector, or
(ii) transfer of perspiration or retained biomarkers from the collector through the window to an internal flow cell for analysis,
wherein the docking station is further configured to reset the collector after analysis by at least one of a rinse, a pressure pulse, or heating.

16. A replaceable microfluidic perspiration collector configured for installation at an inner peripheral surface of a wearable ring, the collector comprising:
an intake region having pores that open to the inner peripheral surface;
a network of microchannels dimensioned to passively wick perspiration by capillary action to at least one reservoir; and
a functional region positioned adjacent to an optical window or a fluidic service port, the functional region including at least one of:
(i) a retention medium selected from ion-exchange material, affinity capture material, or a molecularly imprinted polymer, or
(ii) an indicator or enzyme pad that produces an optical response to a target biomarker,
wherein the collector is configured to be interrogated optically through the optical window or to release fluid through the service port when engaged by a docking station.
